(19) United States
(12) Patent Application Publication
(10) Pub. No.: US 2009/0254122 A1
(43) Pub. Date: Oct. 8, 2009

(54) INTERSPINAL STABILIZATION SYSTEM

(76) Inventor: Sami Khalife, Amiens (FR)

Correspondence Address: Hershkovitz & Associates, LLC, 2845 Duke Street, Alexandria, VA, 22314, US

(21) Appl. No.: 12/307,256

(22) PCT Filed: Jul. 3, 2006
(86) PCT No.: PCT/FR06/01589

§ 371 Date: Dec. 31, 2008

(51) Int. Cl. A61B 17/70

(52) U.S. Cl. 606/249

(57) ABSTRACT

An interspinous stabilization system comprising three elements and characterized in that two of its elements, called spinous anchoring parts (1, 3), are fastened to the superior and inferior spinous processes, and the third element, called an intermediate part (2), is interposed between the other two in order to ensure distraction of the interspinous space and movability of the device in the three spatial planes.

The present invention relates to an item of equipment intended to be implanted at the vertebral level between the spinous processes in order to improve intervertebral stability (by restoring tension on the capsulo-ligamentary apparatus, enlarging the conjugation holes, and reducing pressure in the disc space and between the articular facets) and to avoid, whenever possible, arthrodesis in destabilizing and symptomatic degenerative diseases.

The invention relates more particularly to refinements to this type of implant. Several models of this kind of interspinous implant presently exist, and despite successive improvements developed by manufacturers, none of these implants is entirely satisfactory.

Interspinous implants are generally single-piece, made either of metal or of rigid materials (PEEK, etc.) or of flexible materials (polyurethane covered with woven polyester, or flat woven polyester rolled into a cylinder). Fastening methods are designed in such a way that it is difficult to fasten more than one space at a time, with a tightening tension that is difficult to constantly evaluate. The result is either excessively rigid fastening that can sometimes lead to arthrodesis (which is not the objective), or insufficient fastening that can result in medium-term loosening, with painful conflict between the implant and the spinous processes.

The implant according to the present invention provides an effective solution to the underlying problem of adapting the implant to all anatomical and physiological variations.

The implant according to the present invention comprises three elements: two rigid spinous anchoring parts that can be of variable shapes (identical or not), and one intermediate part of variable thickness and physical consistency, allowing the installation height and elasticity to be controlled at will. The intermediate piece fits between the spinous anchoring parts.

According to one characteristic of the invention, the interfaces between the spinous anchoring parts and the intermediate part are flat or cylindrical in shape, so that upon installation they can be fastened in rotationally movable fashion while ensuring satisfactory and lasting stability.

According to another characteristic of the invention, the implant can be fastened to the spinous processes by a metal device that passes transversely through the bone or by a flexible ligamentary device looping around the processes.

According to an advantageous provision of the invention, the shape of the implant and the fastening methods are designed so that two or more adjacent spaces can be equipped if necessary.

The interspinous stabilization system according to the present invention, comprising three elements, is thus characterized in that two of its elements, called spinous anchoring parts, are fastened to the superior and inferior spinous processes, and the third element, called an intermediate part, is interposed between the other two to ensure distraction of the interspinous space and movability of the device in the three spatial planes.

According to a complementary characteristic, the spinous anchoring parts possess, on the side opposite the spinous process, orifices of various shapes, or blocks, in which the intermediate part fits by way of the corresponding blocks or orifices in order to ensure movability of the system.

According to another characteristic, the intermediate part that is intended to be interposed between the spinous anchoring parts has different shapes and heights and is equipped or not equipped with passthroughs or blocks, and is fabricated with different types of material, to facilitate movement between the various elements of the device.

It may be added that the intermediate part has, for example, an elliptical shape that fits into orifices of the same shape on the spinous anchoring parts in order to provide multidirectional movement for the device.

It may also be noted that the intermediate part is equipped with cylindrical blocks that fit into the corresponding orifices of the spinous anchoring parts.

According to another characteristic, the spinous anchoring parts are equipped with cylindrical orifices to receive the cylindrical blocks of the intermediate part, thus forming a rotationally movable device.

According to a variant, the spinous anchoring parts are equipped with oblong orifices to receive the cylindrical blocks of the intermediate part, thus forming a device that is movable rotationally and in laterally sliding fashion.

According to another variant implementation, the intermediate part is equipped with a threaded anterior orifice to facilitate grasping and assembly, and has transverse tunnels passing through that allow fastening of the system by way of a flexible ligamentary device and provide an additional contribution to the shock-absorbing effect.

It may be added that the intermediate part is constituted by a planar segment to allow rotation and anterior-posterior and lateral translation, and a spherical segment to permit anterior, posterior, and lateral tilting. The entire assembly permits movability in the three spatial planes.

Other characteristics and advantages of the invention will emerge from the description below with reference to the attached drawings, which are provided only as non-limiting examples.

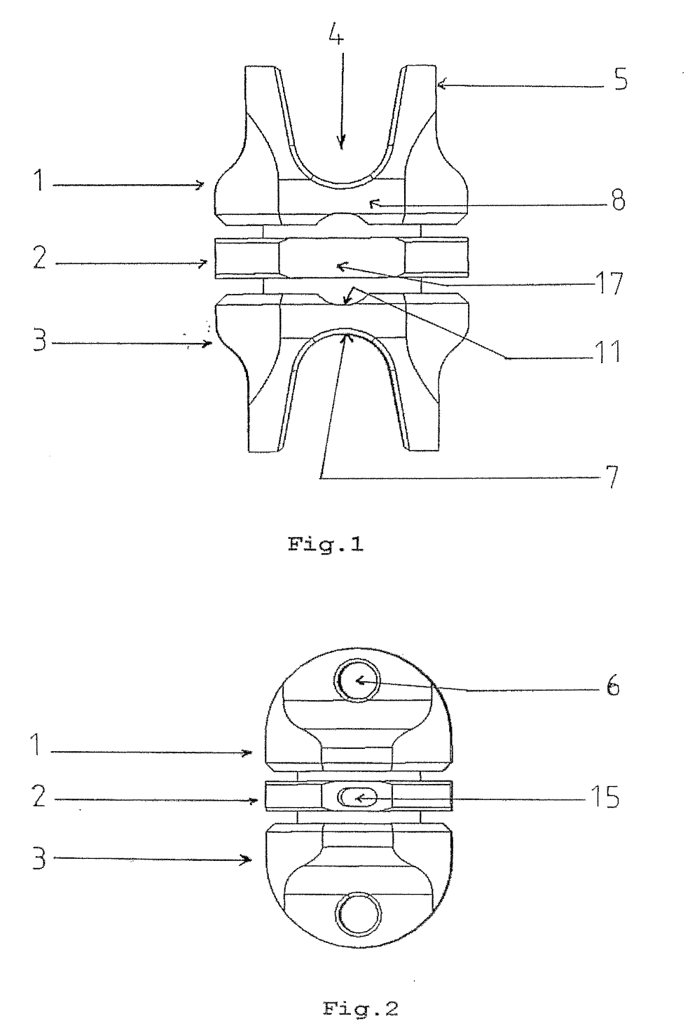

FIGS. 1 to 9 depict the various parts of the system according to the present invention.

FIGS. 1 to 3 illustrate a first embodiment called a “movable system.”

FIG. 1 is a front view of the assembled movable system.

FIG. 2 is a side view of the assembled movable system.

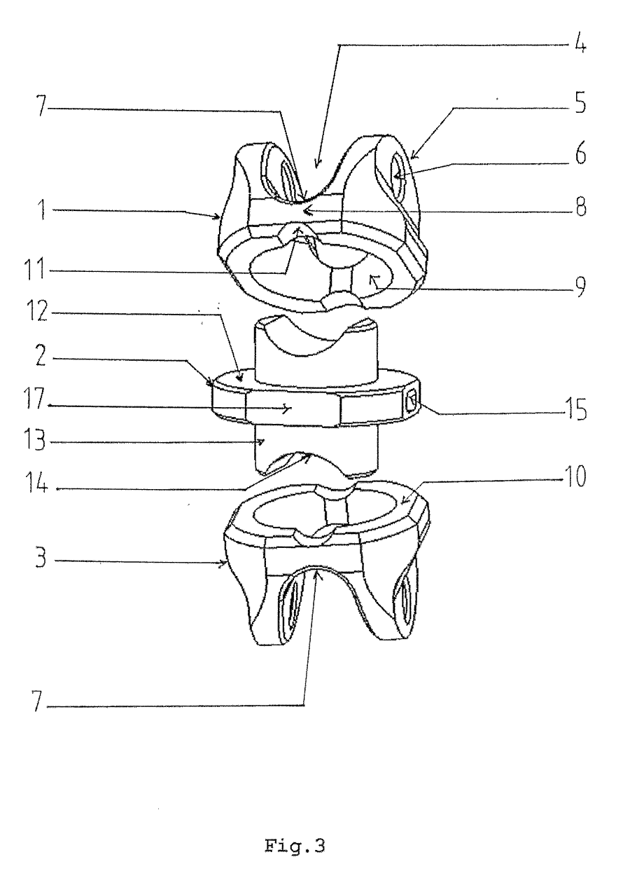

FIG. 3 is a perspective view of the three separated parts of the movable system.

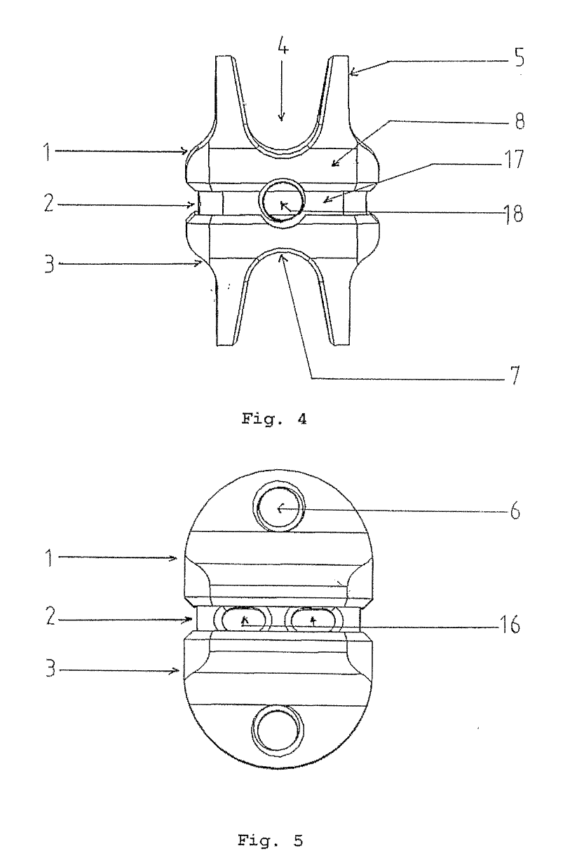

FIGS. 4 to 6 illustrate a second embodiment called a “fixed system.”

FIG. 4 is a front view of the fixed system.

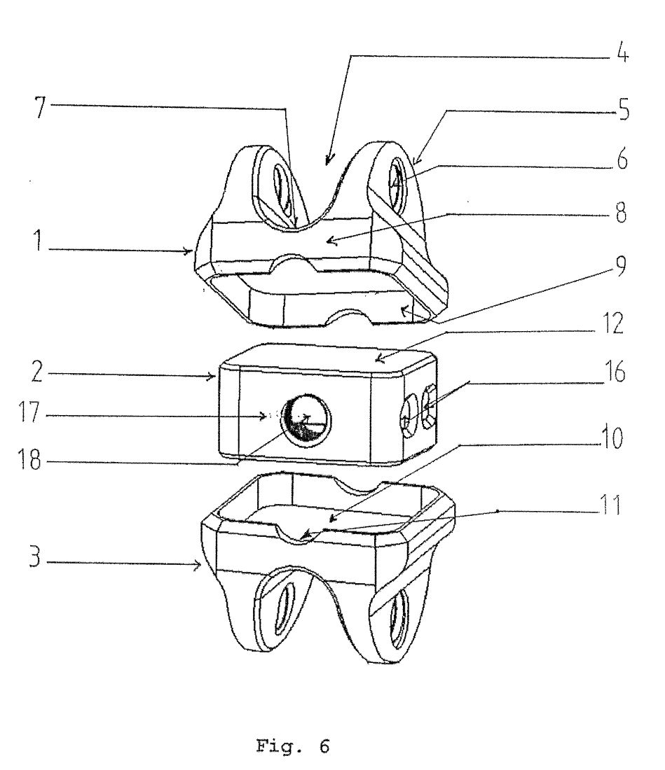

FIG. 5 is a side view of the fixed system.

FIG. 6 is a perspective view of the three parts of the fixed system.

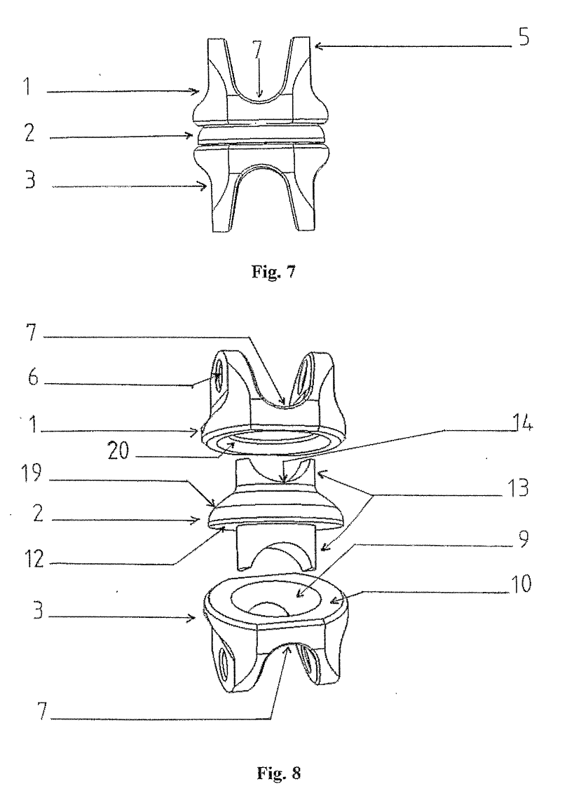

FIGS. 7 to 9 illustrate a third embodiment called a “spherical system.”

FIG. 7 is a front view of the spherical system.

FIG. 8 is a perspective view of the three parts of the spherical system.

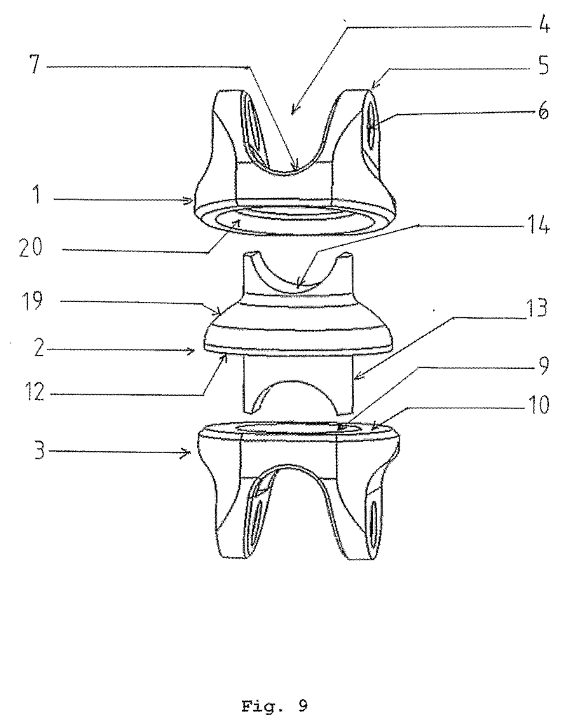

FIG. 9 is another perspective view of the three parts of the spherical system.

The attached drawings illustrate the invention. A description will be given below, with reference to FIGS. 1 to 9, of the characteristics and operation of the “interspinous stabilization system.”

The system according to the present invention comprises three elements: two anchoring parts 1, 3, namely a superior spinous anchoring part 1, an intermediate part 2, and an inferior spinous anchoring part 3.

According to a characteristic of the invention, all the elements are machined using metallic or nonmetallic biocompatible materials, and can be adapted to all anatomical variations.

According to an advantageous design, spinous anchoring parts 1, 3 are made up of a channel-shaped body 4 having a throat 7 of cylindrical section, flared side walls 5 penetrated by two horizontally aligned orifices 6 allowing grasping and fastening using a metal device, the ridges of which are rounded and blunted. Flat base 10 of spinous anchoring parts 1, 3 constitutes, for the first two embodiments, the interface with flat surface 12 of intermediate part 2.

According to a second advantageous design, spinous anchoring parts 1, 3 possess, at their base, orifices 9 of various shapes, or blocks, in which intermediate part 2 will fit by way of corresponding blocks 13 or orifices.

According to an advantageous design, orifices 9 of the spinous anchoring parts can have a section that is square for the fixed model (FIG. 6), circular (FIG. 3) to allow rotational movements, or oblong to allow rotational and laterally sliding movements.

Spinous anchoring parts 1, 3 possess anterior and posterior flats 8 or segments having a median rib 11 that facilitates alignment and assembly.

According to another characteristic of the invention, intermediate part 2 that is intended to be interposed between spinous anchoring parts 1, 3 can be of different shapes and heights, equipped or not equipped with blocks 13 or orifices.

According to an essential characteristic of the invention, intermediate part 2 can be equipped with cylindrical blocks 13 that fit into cylindrical orifices 9 of spinous anchoring parts 1, 3 to form a rotationally movable device. When orifices 9 of spinous anchoring parts 1, 3 are oblong, the device becomes movable rotationally and in laterally sliding fashion.

The ends of blocks 13 of intermediate part 2 comprise a cutout 14 to prevent any conflict with the spinous process.

Intermediate part 2 can be parallelepipedal in shape, having flat fitting segments 17 corresponding to orifices 10 of the same shape in spinous anchoring parts 1, 3.

Intermediate part 2 can have variable mechanical characteristics in order to provide a shock-absorbing effect if necessary.

Intermediate part 2 has horizontal transverse tunnels 15, 16 passing through it, allowing the system to be fastened to the spinous processes by a flexible ligamentary device, and providing an additional contribution to the shock-absorbing effect.

Intermediate part 2 can be equipped with a threaded anterior orifice 18 to facilitate grasping and assembly.

The invention is of course not limited to the implementations described and depicted by way of example, but also encompasses all technical equivalents as well as combinations thereof.

FIG. 9, for example, illustrates a variant that will be called a “spherical system,” according to which coaction between intermediate part 2 and superior spinous anchoring part 1 is effected by way of a spherical contact, by the coaction of contact surface 10 of the intermediate part and contact surface 20 of anchoring part 1, these two surfaces being spherical and of coacting shape.

1. An interspinous stabilization system comprising three elements, wherein two of its elements, called spinous anchoring parts (1, 3), are fastened to the superior and inferior spinous processes, and the third element, called an intermediate part (2), is interposed between the other two in order to ensure distraction of the interspinous space and movability of the device in the three spatial planes.

2. The system according to claim 1, wherein the spinous anchoring parts (1, 3) possess, on the side opposite the spinous process, orifices (9) of various shapes, or blocks, in which the intermediate part (2) fits by way of corresponding blocks (13) or orifices in order to ensure movability of the system.

3. The system according to claim 1, wherein the intermediate part (2) that is intended to be interposed between the spinous anchoring parts (1, 3) has different shapes and heights and is equipped or not equipped with passthroughs or blocks, and is fabricated with different types of material, to facilitate movement between the various elements of the device.

4. The system according to claim 1, wherein the intermediate part (2) has an elliptical shape that fits into orifices of the same shape on the spinous anchoring parts (1, 3) in order to provide multidirectional movement for the device.

5. The system according to claim 1, wherein the intermediate part (2) is equipped with cylindrical blocks (13) that fit into the corresponding orifices (9) of the spinous anchoring parts (1, 3).

6. The system according to claim 1, wherein the spinous anchoring parts (1, 3) are equipped with cylindrical orifices (9) to receive the cylindrical blocks (13) of the intermediate part (2), thus forming a rotationally movable device.

7. The system according to claim 1, wherein the spinous anchoring parts (1, 3) are equipped with oblong orifices (9) to receive the cylindrical blocks (13) of the intermediate part (2), thus forming a device that is movable rotationally and in laterally sliding fashion.

8. The system according to claim 1, wherein the intermediate part (2) is equipped with a threaded anterior orifice (18) to facilitate grasping and assembly, and has transverse tunnels (15, 16) passing through that allow fastening of the system by way of a flexible ligamentary device and provide an additional contribution to the shock-absorbing effect.

9. The system according to claim 1, wherein the intermediate part (2) is constituted by a planar segment to allow rotation and anterior-posterior and lateral translation, and a spherical segment (19) to permit anterior, posterior, and lateral tilting, the entire assembly permitting movability in the three spatial planes.
